(19) United States
(12) Patent Application Publication
(10) Pub. No.: US 2010/0262068 A1
(43) Pub. Date: Oct. 14, 2010

(54) Method for creating an autologous radiofrequency capsule for containing filling solutions

(75) Inventors: Egidio Tranfaglia, Napoli (IT); Valerio Matano, Itri (IT)

Correspondence Address: MODIANO & ASSOCIATI, Via Meravigli, 16, MILANO, 20123, IT

(73) Assignee: PROMOITALIA GROUP S.P.A.

(21) Appl. No.: 12/461,108

(22) Filed: Jul. 31, 2009

(30) Foreign Application Priority Data
Apr. 10, 2009 (IT) NA2009A000017

(51) Int. Cl. A61M 37/00; A61B 18/18

(52) U.S. Cl. 604/21; 604/506; 606/33

(57) ABSTRACT

A method for creating an autologous radiofrequency capsule for containing filling solutions, whose particularity is that it comprises the combined use of bipolar radiofrequencies and of filling or bio-revitalizing solutions based on hyaluronic acid or similar products.

The present invention relates to a method for producing an autologous radiofrequency capsule for containing filling solutions.

### BACKGROUND ART

Anti-aging, lifting, filling and skin blemish elimination therapies are known which employ several methods, among which:
methods for intradermal injection of solutions based on hyaluronic acid;
methods for subcutaneous injections of bio-revitalizing products;
methods for intradermal emissions of a radiofrequency wave;
combined use of hyaluronic acid or similar products and radiofrequency;
invasive methods for filling “large volume” regions;
methods for increasing the circumference of the penis;
methods for treating telangiectasias.

Filling solutions or fillers are injected to fill wrinkles, impressions, scars, or to increase the volume of small or large areas.

Among the most widespread fillers there is hyaluronic acid, which is a component of the dermis and makes the skin elastic and soft.

So-called “bio-revitalization” is a medical treatment aimed at integrating in the dermis substances that the body is no longer able to produce in the adequate form, and in which the injection of hyaluronic acid or of other basic components for reabsorbable fillers is ever more often complemented by products such as vitamins and coenzymes.

Among known methods there is also the method that consists in using a probe that emits an invasive or non-invasive radiofrequency.

The temperature increase caused by the transfer of heat to the tissues by the waves emitted by the probe causes denaturation of the collagen fibers, with immediate contraction and stimulation of the activity of fibroblasts, with an increase in dermal density.

An anti-aging technique provides for the possibility to use simultaneously reabsorbable filler and radiofrequency, which produces an extremely fine ablation with thermal damage and destruction of a volume of tissue in a controlled and reproducible manner.

Filling of the buttocks, of the breast and of post-liposuction cavities is performed predominantly either by inserting prostheses or by initially removing fat and subsequently inserting it in the regions to be filled, or by the infiltration of a particular type of biocompatible gel.

Increase in the circumference of the penis instead can be achieved by means of a surgical procedure that cuts the suspensory ligament of the penis, or by lipopenisculpture, which consists in aspirating autologous fat which is subsequently transplanted or by using a penis elongation device provided with two small telescopic rods, or in the infiltration of biocompatible gel.

Finally, among blemish elimination methods, techniques with sclerosant injections and “vascular” lasers for telangiectasia are noted.

Although the described known types of methods are currently used extensively, they are not devoid of drawbacks.

As regards use of the filler alone, it is noted that it has a limited duration. Reabsorption in fact depends on the speed of action of hyaluronidase as well as on the lifestyle of the patient and leads to the need to repeat the procedure periodically.

As regards injection of bio-revitalizing solution alone, it is noted that its effectiveness and duration are limited, because it causes a poor activation of fibroblasts and because solutions containing hyaluronic acid plus vitamins and/or amino acids and other antioxidants often tend to be easily displaced.

Emission of radiofrequency waves alone, with respect to the filler, does not have an immediate effect, since the denaturation of collagen fibers is followed, over a period of approximately three weeks, by the stimulation of fibroblasts and by the formation of new collagen.

Moreover, the power and frequency ranges usually used often cause traumatic side effects: edema, reddening, erythemas, postoperative dyschromias, scarring, perception of heat mixed with paresthesia of the treated region.

Combination of the simultaneous use of the radiofrequency wave and of hyaluronic acid, as currently commercially available, also suffers obvious drawbacks: the use of high power levels causes thermal damage which, over time, will lead to the creation of a fibrotic channel.

This fibrotic channel is absolutely not useful for the placement of the filler, because it facilitates its swifter degradation; moreover, the parameters used in these devices cause thermal damage, with excessive heating which can lead to significant protein denaturation, rendering the procedure ineffective.

Other than for the reasons mentioned above, these drawbacks also occur with the simultaneous use of radiofrequency wave and of filler injection, which eliminates the possible synergy of the two devices.

These drawbacks can be observed also in the aesthetic surgery techniques currently used to fill large volumes, which have a number of disadvantages.

In particular, the insertion of prostheses is always a highly invasive surgical procedure and also entails a long postoperative hospitalization period and massive side effects.

Insertion of autologous fat in the regions to be filled requires as a necessary condition the presence of excess fat in regions where it can be easily removed, and such fat can also have granularity.

Finally, with infiltration of biocompatible gel alone, the results have a rather limited duration and therefore require frequent touch-ups.

Further drawbacks can be observed in known types of methods currently used to achieve circumferential increase of the penis, which substantially consist in the same drawbacks described for the filling of “large volumes”.

As regards the application of two small telescopic rods to the sides of the penis, this is a technique that requires the user to wear the device for several hours per day for a period of time that can vary from twelve to twenty-four months.

Finally, with infiltration of biocompatible gel alone, the results have a rather limited duration and therefore require frequent touch-ups.

Other drawbacks can be observed in known types of methods currently used for telangiectasia.

In particular, sclerosant injections often have post-sclerotic hyperpigmentation as a side effect.

A laser is instead suitable only for capillaries and small veins up to 1.5-2 mm in diameter. Beyond this order of magnitude, laser treatment requires fluences and spots that make it too painful.

Laser causes physical endotheliitis instead of a chemical one, and can do nothing in terms of reoccurrence of new capillaries. Moreover, the laser damages the epidermis that lies above the treated vessel and causes skin hypochromia.

### SUMMARY OF THE INVENTION

The aim of the present invention is to obviate these drawbacks by providing an aesthetic medicine method for creating an autologous containment capsule that extends and enhances the effect of bio-revitalizing agents and fillers in filling the face, the breast, the buttocks, post-liposuction cavities and the penis without causing thermal damage and thermolysis.

This aim, as well as other objects which will become better apparent hereinafter, are achieved by an aesthetic medicine method for creating an autologous radiofrequency capsule for containing filling solutions, characterized in that it comprises the combined use of bipolar radiofrequencies and filling or bio-revitalizing solutions based on hyaluronic acid or similar products.

### BRIEF DESCRIPTION OF DRAWINGS

Further characteristics and advantages of the present invention will become apparent from the description of a preferred but not exclusive embodiment of the aesthetic medicine method according to the invention, illustrated by way of non-limiting example in the accompanying drawings, wherein:

FIG. 1 is a schematic view of the needle/electrode used with the aesthetic method according to the invention;

FIG. 2 is a view of the operation for 360° rotation in filling facial wrinkles;

FIGS. 3 and 4 are views of the operations related to the filling of the cheekbones by means of the mapping known as F 3 ST;

FIG. 5 is a view of the operations related to breast and buttock filling with the path known as “parachute-style with spoke formation”;

FIG. 6 is a view of the steps related to the filling of the calves and of post-liposuction cavities with the path known as “double tunnel”;

FIG. 7 is a view of the steps related to the filling of the penis or to bio-revitalization with the path known as MITT.

### DESCRIPTION OF PREFERRED EMBODIMENTS

With reference to the figures, the individual steps and operations of the aesthetic medicine method according to the present invention are described hereinafter.

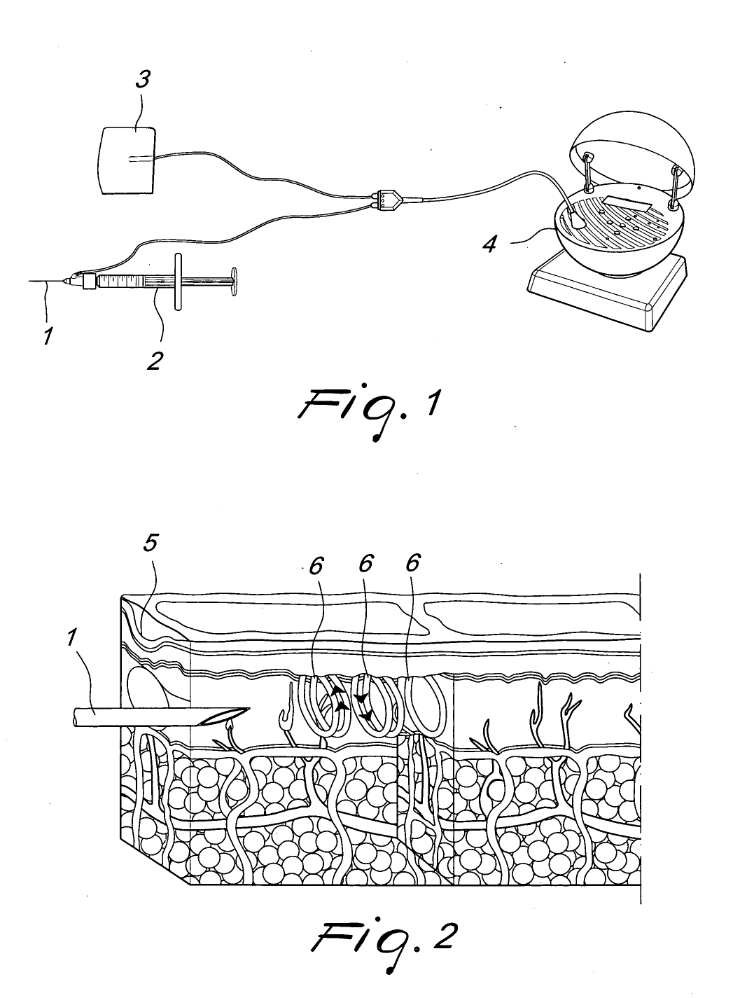

FIG. 1 is a view of the needle/electrode connected to the syringe that contains the filling solution, to the plate to be applied to the patient and to the radiofrequency generator.

FIG. 2 illustrates the 360° rotation of the needle/electrode used to fill the wrinkle. Finally, after application of the plate to the patient, there is the step related to the radiofrequency alone, by means of the insertion and advancement of the needle along the initial path of the region treated for filling face wrinkles with a filler.

Once it has completed the path along the treated region, the needle/electrode performs a 360° rotation so as to create a virtual channel for filling the face, and this 360° rotation is repeated in a point-like manner along the entire path of the treated region.

Subsequently, without ever exiting from the injection site, the needle/electrode follows the path again and releases hyaluronic acid with a retrograde linear technique. Different parameters are used depending on the depth of the wrinkle.

More precisely, these parameters are listed hereinafter:
frequency preferably equal to 1134 kHz, needle/electrode preferably of the 22-G type, maximum power preferably equal to 12 W, filler with high viscosity for deep scars and wrinkles, or for remodeling soft tissues;
frequency preferably equal to 1134 kHz, maximum power comprised preferably between 8 and 10 W and variable depending on the structure of the tissue of the patient, needle/electrode comprised preferably between 25 G and 26 G, medium-viscosity filler for wrinkles and scars of medium depth;
frequency preferably equal to 1769 kHz, needle/electrode preferably of the 30-G type, maximum power comprised preferably between 6 and 8 W, low-viscosity filler for treating thin wrinkles;
frequency preferably equal to 1769 kHz, needle/electrode preferably of the 30-G type, maximum power preferably comprised between 4 and 6 W, low-viscosity filler for bio-revitalization treatment;
frequency preferably equal to 1769 kHz, electric cannula preferably of the 16-G type, maximum power preferably equal to 10 W, high-viscosity filler for filling the cheekbones;
frequency preferably equal to 1769 kHz, electric cannula preferably of the 19-G type, maximum power comprised preferably between 2 and 4 W, very high-viscosity filler for filling the penis;
frequency preferably equal to 1134 kHz, electric cannula preferably of the 14-G type, maximum power preferably equal to 15 W, very high-viscosity filler for breast filling;
frequency preferably equal to 1134 kHz, electric cannula comprised preferably between 12 and 14 G, maximum power preferably equal to 30 W, extremely high-viscosity filler for filling the buttocks;
frequency preferably equal to 1769 kHz, electric cannula preferably of the 14-G type, maximum power preferably equal to 12 W, high-viscosity filler for filling the calf regions;
frequency preferably equal to 1769 kHz, electric cannula preferably comprised between 20 G and 22 G, maximum power preferably equal to 5 W, high-viscosity filler for filling post-liposuction cavities.

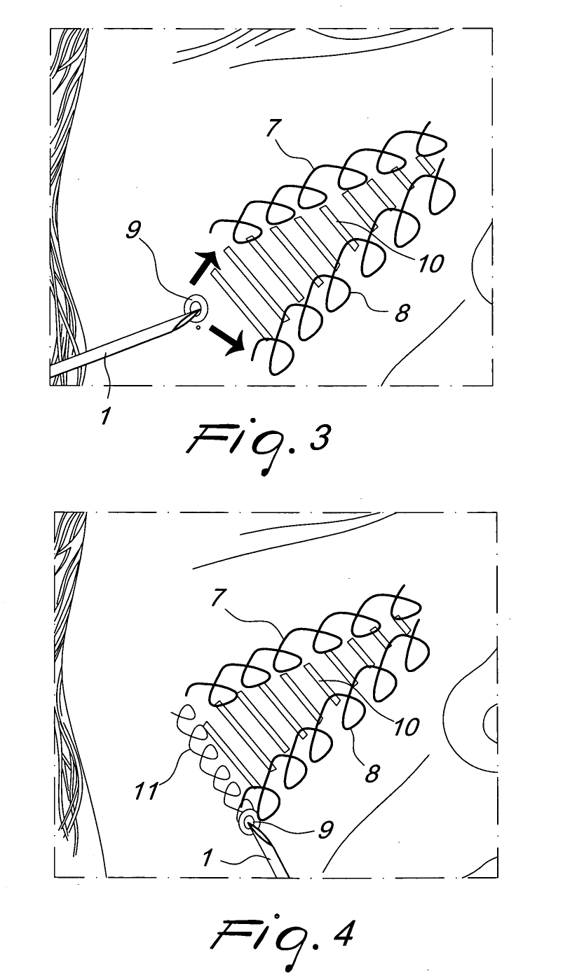

FIG. 3 illustrates the starting points and endpoints of the paths used to correct cheekbones that do not have excessive skin laxness, by means of the mapping known as F 3 ST (acronym for FORENZA 3 STEP TRIANGLE).

The spiral lines relate only to the radiofrequency emission and the shaded line instead relates only to the release of hyaluronic acid.

After placing the plate, the step related to radiofrequency alone occurs, in which an electric cannula with a bevelled tip with a maximum diameter of 14 G is inserted in the injection point, designated by the reference letter A, and spiral channels, designated by the reference letters B and C, are traced by means of a 360° rotation and form the two catheti of a virtual triangle.

Then, without ever exiting from the injection hole, the product is released with a retrograde linear technique along the entire area comprised in the virtual triangle, which is shaded and designated by the reference letter D.

FIG. 4 is a view of the technique used in the case of cheekbones that have excessive skin laxness and the virtual triangle is closed with the spiral channel designated by the reference letter E.

FIG. 5 is a view of the path used to fill the upper quadrant of the breast or buttocks, known as “parachute-style with spoke formation”. After placing of the plate proximately to the area to be treated, the step related to radiofrequency emission alone is performed, in which an electric cannula with a bevelled tip is inserted in a specific point and spiral lines are traced, with emission of the radiofrequency discharge alone and with a 360° rotation, said lines forming the spokes of an imaginary parachute.

Subsequently, without ever exiting from the injection hole, the product is released with a retrograde linear technique along the spaces between the spokes. Advantageously, the different parameters mentioned earlier are used.

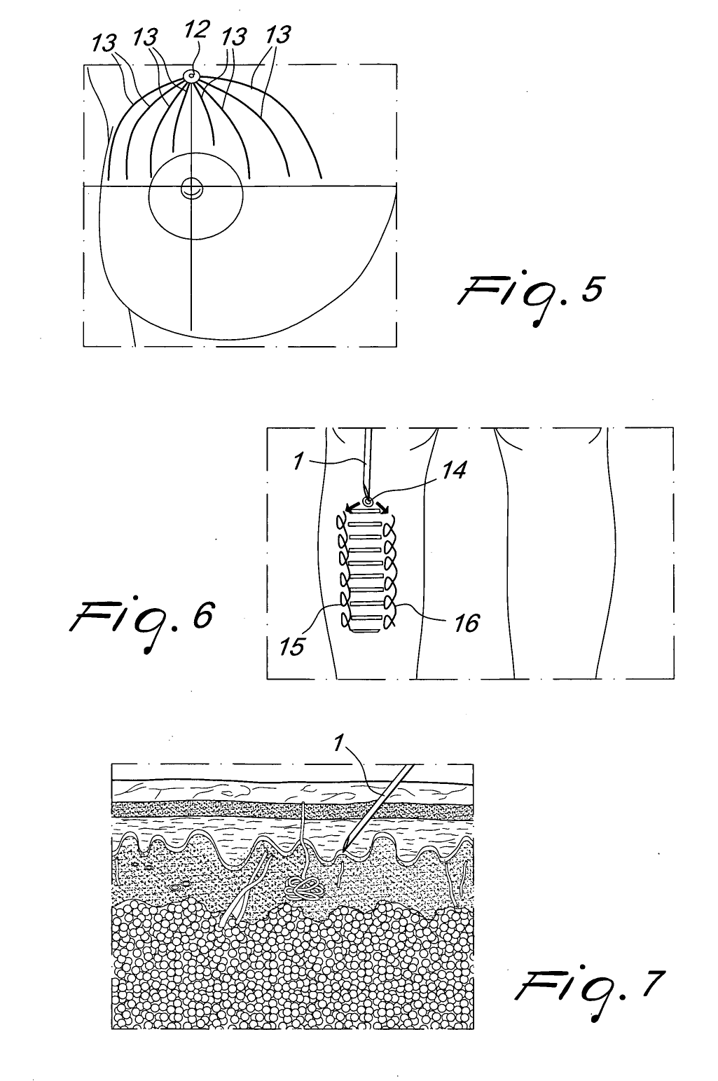

FIG. 6 illustrates the path followed to correct the calves and post-liposuction cavities, known as “parallel tunnel” technique.

After placing of the plate proximately to the area to be treated, the step related to radiofrequency emission alone is performed, in which the needle of the electric cannula is inserted in a specific point and a first tunnel is traced with a 360° rotation.

Then, without ever exiting from the injection hole, a second tunnel is created which is parallel to the preceding one.

Then, without ever exiting from the injection hole, the filler is released into the space created between the two tunnels with a retrograde linear technique. Advantageously, the different parameters indicated earlier are used.

FIG. 7 illustrates the operations for bio-revitalization by MIIT (acronym of MICRO-INTRADERMAL INJECTION TECHNIQUE).

MIIT entails first placing the plate and emitting the RF with retrograde linear technique. Then, after going backwards with the needle along the previous path with the same retrograde linear technique, the solution is released with serial injections.

MITT is applied also in treatment of the penis. In this case, first the plate is positioned and the radiofrequency discharge alone is emitted in the apex of the preputial lamina, so as to simulate a mushroom-like feature with MITT technique.

After going back with the needle along the previous path with the same retrograde linear technique, the solution is released. Advantageously, the various parameters indicated earlier are used.

In telangiectasia, instead, after the placing of the plate on the skin, a 30-G needle is inserted within the capillary, emission of the 1769-kHz radiofrequency is performed with a maximum power of 4 W, the micro-droplet of sclerosant is emitted, and finally constant radiofrequency emission is performed along the path of the capillary.

In practice it has been found that the method according to the invention fully achieves the intended aim and objects, since it makes it possible to overcome the problems of the background art, entailing several evident advantages.

Other than with traditional techniques, a micro-heating action, without thermal damage or thermolysis, is in fact obtained.

More precisely, a reactive process is performed which is such as to create a repairing “scarring” reaction, which subsequently creates an autologous containment “capsule” for the filler without fibrosis.

Another advantage of the method according to the invention is that by means of the combined but not simultaneous use of the radiofrequency wave and of the filler, it allows the initial repairing process with subsequent creation of a containment capsule.

A further advantage of the method according to the invention is that a considerable reduction of the side effects of traditional methods is achieved by the use of medium frequencies (PRFR) and of power levels not exceeding 12 W.

Other advantages of the method according to the invention consist in that the duration of the filler and of the bio-revitalizing agents is extended, a considerably smaller amount of filler and bio-revitalizing agent is used, and the effects of the filler or of the bio-revitalizing agent and of the radiofrequency are added together synergistically with immediate effect.

In the case of techniques for filling “large volumes” and the penis, the method according to the present invention leads to a result that is durable over time, with a mini-invasive technique, nearly complete lack of side effects, lack of any type of granularity and a considerable saving effect of product used.

Moreover, this is a natural method, with easy implantation, lack of invasive surgery and lack of scars.

In the case of telangiectasia, the proposed technique makes it possible to perform treatments that are far more effective, since it combines synergistically focused radiofrequency and sclerotherapy.

More precisely, sclerotherapy closes the system of larger upstream vessels, whereas radiofrequency seals effectively the smaller and superficial vessels and prevents small hemorrhages with consequent formation of brown stains.

The use of a needle/electrode directly in the opening of the capillary allows a far more selective coagulation than laser and therefore avoids damage of the epidermis that lies above the treated vessel and avoids the formation of skin hypochromia.

Radiofrequency is delivered by use of the needle/electrode connected to the syringe that contains the filler solution, to the plate and to the radiofrequency generator.

Such needle/electrode, inserted within the area to be treated, emits an alternating electron stream, which is concentrated on the tip and creates a weakelectromagnetic field that is irradiated for a few millimeters, two to three, within the treated region.

The needle/electrode, by generating minute electrical oscillations within the tissue, provides a physical mechanism for energy transfer.

The bipolar electric wave, to be placed a few centimeters away from the area, travels through a circuit in which the poles are arranged on the tip of the needle and on a plate, respectively, and ensures a bidirectional flow of current.

This wave is emitted with a variable power level that does not exceed 30 W and at two different frequencies, one preferably equal to 1134 kHz and the other one preferably equal to 1769 kHz, and strikes the tissue, producing energy transport at a high concentration on a micro-area.

Micro-heating is thus generated without thermolysis or thermal damage and can induce a reactive process that causes a repairing “scarring” reaction.

This reaction is used subsequently to contain the filler or the bio-revitalizing agent that is injected after the emission of the wave by the same electrode, thus creating an autologous “capsule” for containing the filling material.

The body responds to the electrical discharge, giving rise to a chemotactic leucocyte activation, which becomes an extremely important source of preformed or newly synthesized chemical mediators, but also of growth factors and enzymes.

Moreover, neutrophils produce cytokines, chemokines and growth factors for connective cells, including fibroblast growth factor (FGF) 1, 2 and 5, the expression level of which increases approximately 10-foldin the repairing tissue.

The repairing step is further enhanced by the arrival of macrophages, cells which are even more important than neutrophils because besides being a source of growth and cell modulation they produce TGF-b, PDGF and insulin-like growth factor.

Of these growth factors, TGF-b is probably the most important in the repair process, due to its powerful effects on all the cells involved in this process and not only on fibroblasts, thus stimulating their proliferation and promoting the formation of the granulation tissue from which the capsule for containing the filling solution will then originate.

Another key factor are matrix metalloproteases, a set of enzymes that degrade connective tissue, with a key role in inflammation and in tissue repair.

During the process, these enzymes are subjected to an accurate activation/deactivation process in order to allow the remodeling of the newly formed tissue and the formation of the containment channel.

The method thus conceived is susceptible of numerous modifications and variations, all of which are within the scope of the inventive concept. All the details may further be replaced with other technically equivalent elements.

The disclosures in Italian Patent Application no. NA2009A000017, from which this application claims priority, are incorporated herein by reference.

What is claimed is:

1. A method for creating an autologous radiofrequency capsule for containing filling solutions, comprising the combined use of bipolar radiofrequencies and filling or bio-revitalizing solutions based on hyaluronic acid or similar products.

2. The method according to claim 1, comprising the use of a radiofrequency generator with a maximum power of substantially 30 W which delivers a radiofrequency, releasing the filling solution contained in a syringe to which it is connected at at least two different frequencies, one equal to 1134 kHz and another one equal to 1769 kHz, by means of a connector with two outlets which is connected by wire means at one end to the plate placed on the patient and at the other end to a needle/electrode or an electric cannula.

3. The method according to claim 1, comprising the use of the following parameters, which can vary with the depth of the skin blemish, the product used and with the treated region:
frequency equal to 1134 kHz, 22-G needle/electrode, maximum power 12 W, filler with high viscosity for deep scars and wrinkles, or for remodeling soft tissues;
frequency equal to 1134 kHz, maximum power comprised between 8 and 10 W and variable depending on the structure of the tissue of the patient, needle/electrode comprised between 25 G and 26 G, medium-viscosity filler for wrinkles and scars of medium depth;
frequency equal to 1769 kHz, 30-G needle/electrode, maximum power comprised between 6 and 8 W, low-viscosity filler for treating thin wrinkles;
frequency equal to 1769 kHz, 30-G needle/electrode, maximum power comprised between 4 and 6 W, low-viscosity filler for bio-revitalization treatment;
frequency equal to 1769 kHz, 16-G electric cannula, maximum power equal to 10 W, high-viscosity filler for filling the cheekbones;
frequency equal to 1769 kHz, 19-G electric cannula, maximum frequency comprised between 2 and 4 W, very high-viscosity filler for filling the penis;
frequency equal to 1134 kHz, 14-G electric cannula, maximum power equal to 15 W, very high-viscosity filler for breast filling;
frequency equal to 1134 kHz, electric cannula comprised between 12 and 14 G, maximum power equal to 30 W, extremely high-viscosity filler for filling the buttocks;
frequency equal to 1769 kHz, 14-G electric cannula, maximum power equal to 12 W, high-viscosity filler for filling the calf regions;
frequency equal to 1769 kHz, electric cannula comprised between 20 G and 22 G, maximum power equal to 5 W, high-viscosity filler for filling post-liposuction cavities.

4. The method according to claim 3, wherein, in the case of treatments of facial wrinkles, the electrical radiofrequency discharge and the injection of hyaluronic acid are applied by means of the following operations:
placement of the plate on the skin proximate to the area to be treated,
insertion of the needle along the path of the area to be treated inside the dermis, at a variable depth depending on the type of therapeutic indication,
delivery of the radiofrequency discharge and simultaneous execution of a 360° rotation of the needle so as to create a virtual channel,
repetition of the 360° rotation of the needle in a point-like manner along the entire path of the area to be treated and simultaneous dispensing of the radiofrequency discharge,
interruption of the radiofrequency discharge and repositioning of the needle along the path of the area to be treated without ever exiting from the injection site,
release of the filling solution with retrograde linear technique, using the connected syringe, after the path of the area to be treated has been completed, release is performed.

5. The method according to claim 4, wherein, in the case of treatments concerning the filling of the cheekbones, the electrical radiofrequency discharge and the hyaluronic acid injection are applied by means of the technique known as F 3 ST, by means of the following operations:
placement of the plate on the skin proximate to the area to be treated,
insertion of the needle in a precise point,
tracing, by means of a 360° rotation and with radiofrequency emission alone, of spiral channels that form the two catheti of a virtual triangle,
release with retrograde linear technique of the product along the entire area comprised within the virtual triangle without ever exiting from the injection hole.

6. The method according to claim 5, wherein, in the case of cheekbones that have excessive skin laxness, the virtual triangle is closed with a spiral base.

7. The method according to claim 1, wherein the electrical radiofrequency discharge and the injection of filler for filling the breast and the buttocks are applied by means of the parachute-style path with spoke formation, which consists of the following operations:
placement of the plate on the skin proximate to the area to be treated,
insertion of the needle of the electric cannula in a specific point,
tracing, with emission of the radiofrequency discharge alone, with 360° rotation of the tip of the needle, of spiral lines that form the spokes of an imaginary parachute,
release of the product with a retrograde linear technique along the spaces between the spokes without ever exiting from the injection hole.

8. The method according to claim 2, wherein the electrical radiofrequency discharge and the injection of filler to fill the calves and post-liposuction cavity regions are applied by means of the parallel tunnel path, consisting of the following operations:
placing the plate on the skin proximate to the area to be treated,
inserting the needle of the electric cannula in a specific point,
tracing, with emission of the radiofrequency discharge alone, with a 360° rotation technique, of a first tunnel,
creation of a second tunnel, which is parallel to said first tunnel without ever exiting the injection hole,
release of the filler into the space created between the two tunnels with retrograde linear technique without ever exiting from the injection hole.

9. The method according to claim 8, wherein a micro-technique of parallel tunnels is performed in the post-liposuction cavity regions.

10. The method according to claim 2, wherein the electrical radiofrequency discharge and the injection of bio-revitalizing products are applied by means of MIIT, which consists of the following operations:
placing the plate on the skin proximate to the area to be treated and emitting radiofrequency alone, with retrograde linear technique,
releasing the solution with serial injections after the return of the needle along the previous path with the same retrograde linear technique.

11. The method according to claim 2, wherein in penis filling the MIIT path is followed by means of the following operations:
placing the plate on the skin proximate to the area to be treated,
emitting the radiofrequency discharge alone in the apex of the preputial laminae, so as to simulate a mushroom-like feature with MITT technique,
release of the solution after the retraction of the needle along the previous path with the same retrograde linear technique, release is performed.

12. A method for eliminating telangiectasia of variable diameter, without damaging the surrounding epidermis and without producing post-sclerotic hyperpigmentation, comprising the synergistic use of bipolar radiofrequency and of sclerosant solution.

13. The method according to claim 12, comprising a first radiofrequency and then the injection of sclerosant products by means of the following operations:
placement of the plate on the skin proximate to the area to be treated,
insertion of the 30-G needle within the capillary with emission of a radiofrequency equal to 1769 kHz and a maximum power of 4 W,
emission of the micro-droplet of sclerosant,
constant emission of radiofrequency along the path of the capillary.
